(19) United States
(12) Patent Application Publication
(10) Pub. No.: US 2025/0134755 A1
(43) Pub. Date: May 1, 2025

(54) MASSAGE THERAPY DEVICE

(71) Applicant: SHENZHEN KAIYAN MEDICAL EQUIPMENT CO., LTD, Shenzhen (CN)

(72) Inventor: ALAIN DIJKSTRA, Amstelveen (NL)

(73) Assignee: SHENZHEN KAIYAN MEDICAL EQUIPMENT CO., LTD, Shenzhen (CN)

(21) Appl. No.: 18/533,236

(22) Filed: Dec. 8, 2023

(30) Foreign Application Priority Data
Oct. 30, 2023 (CN) 2023229164004

(51) Int. Cl. A61H 15/00; A61H 15/02; A61H 23/02; A61N 1/04; A61N 5/06; H02J 50/10

(52) U.S. Cl. CPC A61H 15/0085; A61H 15/02; A61H 23/0254; A61N 1/0472; A61N 5/0613; H02J 50/10; A61H 2015/005; A61H 2015/0071; A61H 2201/0207; A61H 2201/0214; A61H 2201/10; A61H 2201/123; A61H 2201/1635; A61H 2201/165; A61H 2201/5058; A61H 2201/5097; A61N 2005/0645; A61N 2005/0652; A61N 2005/0659; A61N 2005/0663

(57) ABSTRACT

A massage therapy device includes a housing provided with one or more holes, one or more spherical structures provided partially within the one or more respective holes of the housing, wherein the one or more spherical structures are configured to rotate with respect to the housing, while remaining attached within the housing, and a plurality of stimulation elements disposed along outer surfaces of the one or more spherical structures, wherein the plurality of stimulation elements are directed in an outward direction from the outer surfaces of the one or more spherical structures.

### TECHNICAL FIELD

The present invention generally relates to therapeutic devices. More specifically, the present invention relates to stimulation-based massage therapy devices.

### BACKGROUND ART

Massage therapy devices have been known in the art for some time now. Such massage therapy devices are generally specific for a specific part of the body, such as the face, leg, neck, waist etc., and are therefore designed in different shapes. One device for a specific part of the body cannot conform to other parts of the body. Therefore, for different parts of the body, a user needs to purchase multiple massage therapy devices leading to additional costs. Moreover, common body massagers work by different methodologies such as vibrations, hand hammers, peristaltic extrusion, and other methods which can cause a general feeling of inconvenience to the user. In addition, conventional massage therapy devices only focus on massage therapy and do not combine other therapies such as light therapy, heating, cooling, and electrical current based therapies with massage therapy.

Therefore, there is a need for a device that overcomes the disadvantages and limitations associated with the prior art and provides a more satisfactory solution.

### OBJECTS OF THE INVENTION

Some of the objects of the invention are as follows:

An object of the invention is to provide a massage therapy device that can conform to several parts of the body.

Another object of the invention is to provide a massage therapy device that combines massage therapy with light therapy and other additional therapies such as heating, cooling, and electrical stimulation, etc.

Another object of the invention is to provide a massage therapy device that includes several rolling spherical structures to provide massage therapy to the several body parts of the user.

Another object of the invention is to provide a massage therapy device where several stimulation elements are attached to the spherical structures for providing stimulations such as light therapy, heating effect, cooling effect, very small magnitudes of electrical current to the several body parts of the user.

Yet another object of the invention is to provide a massage therapy device where the several stimulation elements can be powered wirelessly with or without the presence of batteries in the rolling balls.

### SUMMARY OF THE INVENTION

According to a first aspect of the present invention, there is provided a massage therapy device. The massage therapy device includes a housing provided with one or more holes, one or more spherical structures provided partially within the one or more respective holes of the housing, wherein the one or more spherical structures are configured to rotate with respect to the housing, while remaining attached within the housing, and a plurality of stimulation elements disposed along outer surfaces of the one or more spherical structures, wherein the plurality of stimulation elements are directed in an outward direction from the outer surfaces of the one or more spherical structures.

In one embodiment of the invention, the housing is configured to receive power through wired or wireless means from a power source.

In one embodiment of the invention, the housing includes a wireless charging coil and the one or more spherical structures include one or more respective induction coils, the one or more induction coils being configured to receive power at any angle or orientation through coupling with the electromagnetic field generated by the wireless charging coil due to flow of electric current in the wireless charging coil.

In one embodiment of the invention, the one or more spherical structures include one or more respective batteries configured to be charged through the power received by one or more respective induction coils.

In one embodiment of the invention, the power received by the one or more induction coils is used to activate the plurality of stimulation elements of the one or more spherical structures.

In one embodiment of the invention, the one or more spherical structures include one or more respective balancing devices configured to align the one or more respective induction coils with the wireless charging coil of the housing when the massage therapy device is in a stationary position.

In one embodiment of the invention, the plurality of stimulation elements includes one or more of Light Emitting Diodes (LEDs), heating elements, cooling elements, vibration elements, electrodes and combinations thereof.

In one embodiment of the invention, the LEDs are configured to emit radiation in visible light and infrared frequencies of the electromagnetic spectrum.

In one embodiment of the invention, the wavelengths of the light emitted by the LEDs lie in the ranges of 380 nm to 700 nm for the visible light and 845 nm to 855 nm for the infrared light.

In one embodiment of the invention, the one or more spherical structures are made up of himalayan salt thereby allowing radiation from the LEDs to pass through.

In one embodiment of the invention, the massage therapy device further comprises one or more gyroscope sensors provided within the one or more respective spherical structures.

In one embodiment of the invention, the gyroscope sensor in each ball is configured to disconnect power supply to the plurality of stimulation elements that are directed towards the ambient, when the massage therapy device is in use.

In one embodiment of the invention, the one or more spherical structures are removably attached to the housing and are replaceable with respect to the housing.

In one embodiment of the invention, the massage therapy device further comprises a control unit configured to control the operation of the plurality of stimulation elements in response to input received from an external communication device.

According to a second aspect of the present invention, there is provided a method of manufacturing a massage therapy device, the method comprising providing a housing with one or more holes, providing, partially, one or more spherical structures in the one or more respective holes, wherein the one or more spherical structures are configured to rotate with respect to the housing, while remaining attached within the housing, and disposing a plurality of stimulation elements along outer surfaces of the one or more spherical structures, wherein the plurality of stimulation elements are directed in an outward direction from the outer surfaces of the one or more spherical structures.

In the context of the specification, the term “processor” refers to one or more of microprocessors, a microcontroller, a general-purpose processor, a Field Programmable Gate Array (FPGA) or an Application Specific Integrated Circuit (ASIC), and the like.

In the context of the specification, the phrase “storage memory” refers to one or more of a volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc., or a non-volatile storage memory such as EPROM, EEPROM or flash memory or the like.

In the context of the specification, the phrase “communication interface” refers to a device or a module enabling direct connectivity via wires and connectors such as USB, HDMI, VGA, or wireless connectivity such as Bluetooth or Wi-Fi or Local Area Network (LAN) or Wide Area Network (WAN) implemented through TCP/IP, IEEE 802.x, GSM, CDMA, LTE or other equivalent protocols.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of this specification, terms like “light”, “radiation”, “irradiation”, “emission” and “illumination”, etc. refer to electromagnetic radiation in wavelength ranges varying from the visible light wavelengths (380-700 nm) to Infrared (IR) wavelengths (780 nm-1 mm), wherein the range is inclusive of visible light and IR wavelengths. The IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, “Light Emitting Diodes (LEDs)” are envisaged to be characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow; blue and white colors), Surface Mount LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley. J. N (2004). “International OLED Technology Roadmap”. IEEE Journal of Selected Topics in Quantum Electronics. Vol. 10. No. 1. that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2, and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a massage therapy device in accordance with an embodiment of the present invention;

FIG. 1B illustrates an exploded view of the massage therapy device of FIG. 1A;

FIG. 2 illustrates a sectional view of the massage therapy device in accordance with an embodiment of the present invention;

FIG. 3 illustrates a user using a massage therapy device, in accordance with another embodiment of the present invention; and

FIG. 4 illustrates a method of manufacturing a massage therapy device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

It is envisaged that a massage therapy device is provided that can conform to several body parts of a user and additionally provide other kinds of stimulation such as light therapy, heating, cooling, and electrical stimulation. In that regard, the massage therapy device may include several spherical ball-like structures that can conform to the several body parts of a user. The spherical structures may be powered using wireless charging through the housing and optionally may include batteries. Further, balancing devices may be provided in the spherical structures that may keep them aligned with a wireless charging coil of the housing.

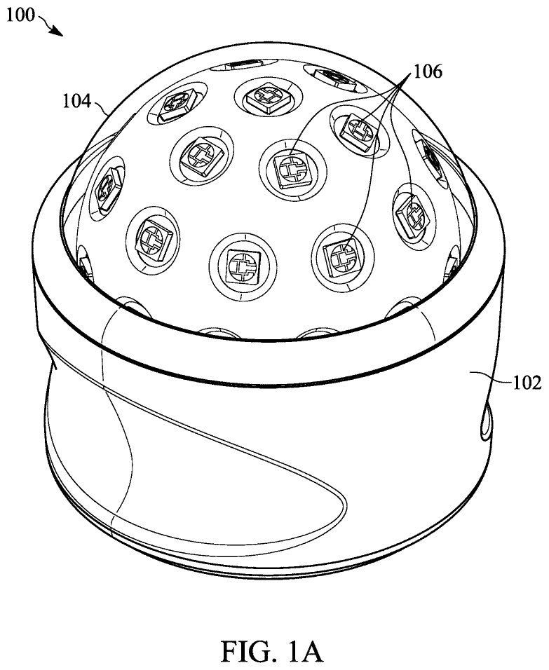

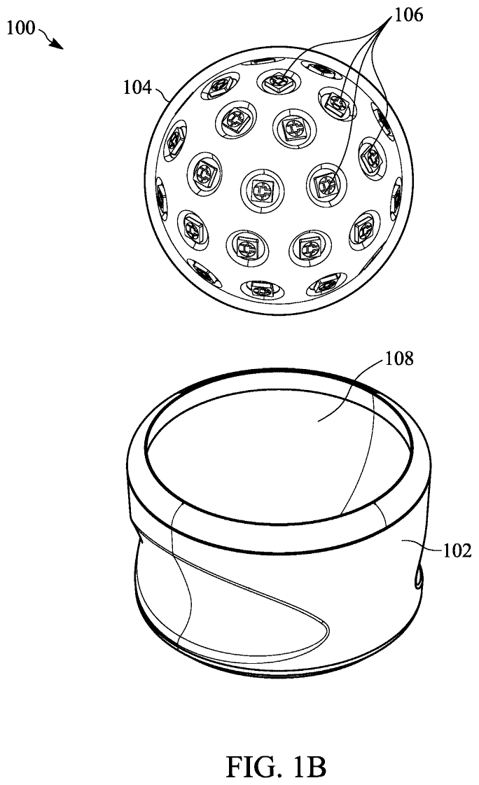

FIG. 1A illustrates a perspective view of a massage therapy device 100 (hereinafter referred to as “the device 100”) in accordance with an embodiment of the present invention. FIG. 1B illustrates an exploded view of the massage therapy device of FIG. 1A. Referring to FIGS. 1A and 1B, the device 100 includes a housing 102 with a hole 108. In several embodiments of the invention, the housing 102 may have more than one hole 108. The housing 102 may be made up of a polymeric material such as Low Density Polyethylene (LDPE), High Density Polyethylene (HDPE), Polyvinyl Chloride (PVC), and Acrylonitrile Butadiene Styrene (ABS) etc. Further, a spherical structure 104 has been partially disposed in the hole 108 of the housing 102. In the embodiments where the housing 102 includes more than one hole 108, one spherical structure 104 may be disposed in each one of the holes 108. The spherical structure 104 is configured to rotate with respect to the housing 102, while remaining attached within the housing 102. The rotation of the spherical structure 104 with respect to the housing 102 allows the device 100 to conform to several parts of the body. In one embodiment of the invention, the spherical structure 104 is removably attached with the housing 102 and is replaceable with respect to the housing 102. In that regard, the spherical structure 104 may be attached with the housing 102 with the help of fastners or through snap fit arrangement. The removable attachment of the spherical structure 104 would allow cleaning and repair/replacement of the spherical structure 104, as well as the housing 102.

Further, a plurality of stimulation elements 106 have been disposed along outer surface of the spherical structure 104. In several embodiments of the invention, the plurality of stimulation elements 106 include one or more of Light Emitting Diodes (LEDs), heating elements, cooling elements, vibration elements, electrodes, and combinations thereof. The LEDs are configured to emit radiation in visible light and infrared frequencies of the electromagnetic spectrum. In other words, the wavelengths of the light emitted by the LEDs lie in the ranges of 380 nm to 700 nm for the visible light and 845 nm to 855 nm for the infrared light. Further, the LEDs are configured to operate in pulsed or continuous mode of operation. In several embodiments of the invention, the spherical structure 104 is made up of himalayan salt. Since himalayan salt is essentially transparent, the radiation from the LEDs would be able to pass through the spherical structure 104. The heating elements are selected from a group consisting of metal heating elements, ceramic heating elements, semiconductor heating elements, thick film heating elements, polymer based heating elements, composite heating elements, and combination heating elements. The cooling element may be a thermoelectric cooler, also known as a Peltier heat pump. The vibration elements may include eccentric rotating motors or linear resonant actuators.

The plurality of stimulation elements 106 are directed in an outward direction from the outer surface of the spherical structures 104. The outward direction of the plurality of stimulation elements 106 ensures that stimulation effect from the the plurality of stimulation elements 106 is fully absorbed by the body of a user. In several embodiments of the invention, the spherical structure 104 includes a gyroscope sensor for determination of the orientation of the spherical structure 104. The signals from the gyroscope sensors are used to disconnect power supply to the plurality of stimulation elements 106 that are directed towards the ambient or the inner surfaces of the housing 102, when the device 100 is in use. In this regard, when some of the plurality of stimulation elements 106 are directed towards the ambient, then those are not directed towards the skin of the user, and this may lead to the wastage of power if the electrical current supplied to those stimulation elements are not causing any effect on the body of the user. Therefore, disconnecting the power supply to the plurality of stimulation elements 106 that are directed towards the ambient or directed away from the skin of the user saves power that would be lost to the ambient.

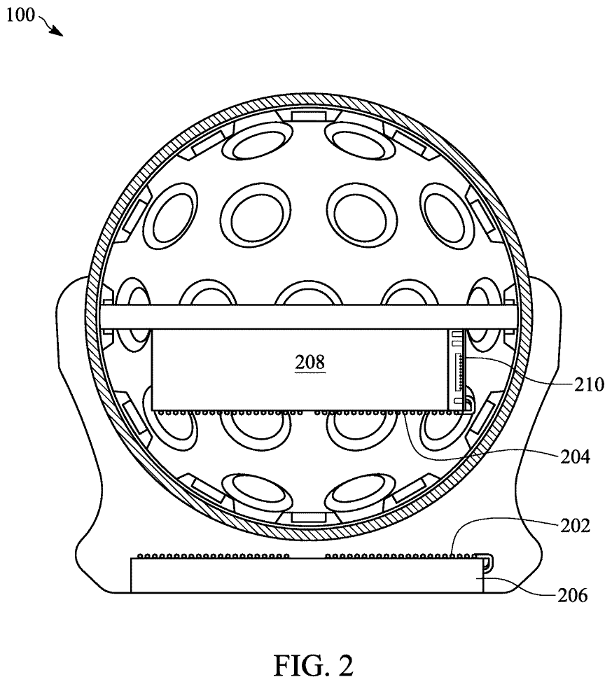

FIG. 2 illustrates a sectional view of the device 100 in accordance with an embodiment of the present invention. The housing 102 is configured to receive power through wired or wireless means from a power source 206. The power source 206 may be a DC power source such as battery. The battery may be rechargeable or non-rechargeable battery. The rechargeable battery may be selected from a group consisting of Nickel-Metal-Hydride battery, Lithium-Ion battery, or a Lithium-polymer battery. The power source 206 may also be an AC power source, such as a domestic power supply. Further, the housing 102 includes a wireless charging coil 202. The wireless charging coil 202 receives the power being supplied to the housing 102 and generates an electromagnetic field around the wireless charging coil 202. Also, the spherical structure 104 includes an induction coil 204. The induction coil 204 is configured to receive power at any angle or orientation through coupling with the electromagnetic field generated by the wireless charging coil 202, due to the flow of electric current in the wireless charging coil 202.

In several embodiments of the invention, the spherical structure 104 includes a battery 208, and the plurality of stimulation elements 106 receives power from the battery 208. In several alternate embodiments of the invention, the induction coil 204 directly powers the plurality of stimulation elements 106. In several embodiments of the invention, the spherical structure 104 includes a balancing device 210 configured to align the induction coil 204 with the wireless charging coil 202 of the housing 102 when the device 100 is in a stationary position. The design and functioning of the balancing device 210 have been described in the United States Patent Publication Numbered US 20200315907 A1 which is incorporated herein by reference in its entirety. This would allow a more efficient electromagnetic coupling between the wireless charging coil 202 and the induction coil 204.

In several embodiments of the invention, the device 100 further includes a control unit configured to control the operation of the plurality of stimulation elements 106 in response to an input received from an external communication device. The external communication device may be selected from a group consisting of a cellphone, a tablet PC, a desktop PC, a notebook PC, and the like. In one embodiment of the invention, the control of operation may include modifying irradiation characteristics such as mode, frequency and wavelengths of the LEDs. In another embodiment of the invention, the control of operation may include modifying the heating or cooling temperatures of the heating and cooling elements. In another embodiment, the control of operation may include modifying vibration characteristics of the vibrator. The control unit in that regard may include a processor, a memory unit and a communication interface. The memory unit may include machine readable instructions for execution by the processor. Further, the communication interface may allow the control unit to communicate with the external communication device.

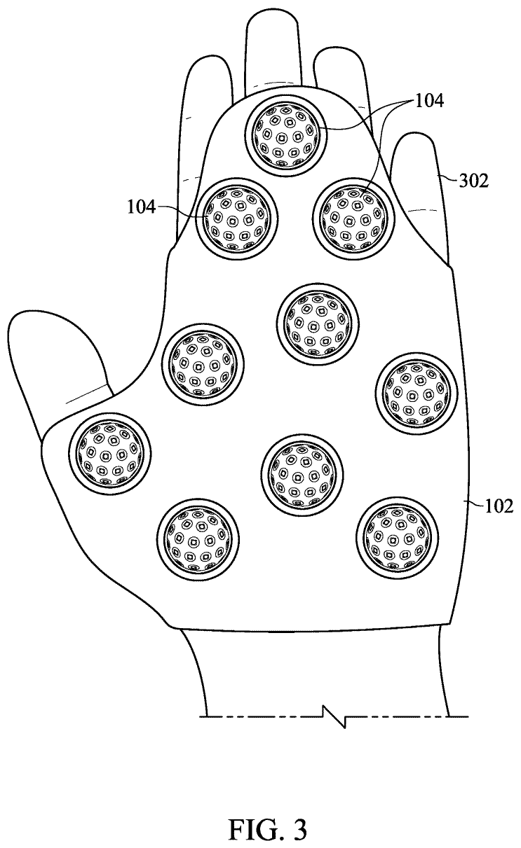

FIG. 3 illustrates a user 302 using the device 100, in accordance with another embodiment of the present invention. As illustrated in FIG. 3, the housing 102 includes more than one holes 108 and more than one spherical structures 104 have been disposed in the respective holes 108. Further, the housing 102 embodies a shape of a hand glove.

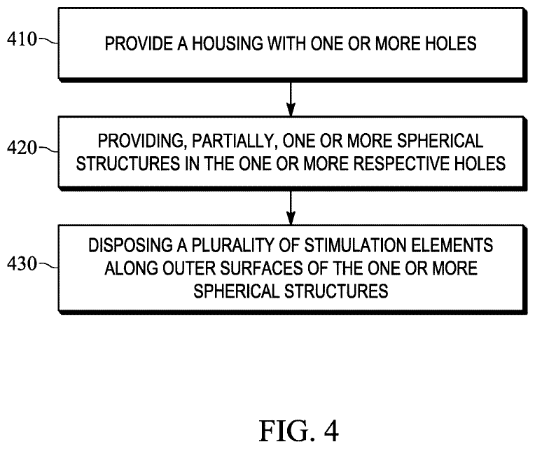

FIG. 4 illustrates a method 400 of manufacturing the device 100, in accordance with an embodiment of the present invention. At step 410, the housing 102 is provided. The housing 102 may be made up of a polymeric material such as LDPE, HDPE, PVC, ABS and the like. The housing 102 may include one or more holes 108. At step 420, one or more spherical structures 104 are partially provided within the one or more respective holes 108 of the housing 102. The one or more spherical structures 104 are configured to rotate with respect to the housing 102. In several embodiments of the invention, the one or more spherical structures 104 are detachably attached with the housing 102. At step 430, the plurality of stimulation elements 106 are disposed along outer surfaces of the one or more spherical structures 104. The plurality of stimulation elements 106 are directed in an outward direction from the outer surfaces of the one or more spherical structures 104.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing the broadest scope consistent with the principles and the novel and inventive features disclosed or suggested herein. Accordingly, the invention is anticipated to hold on to all other such alternatives, modifications, and variations that fall within the scope of the present invention and appended claims.

1. A massage therapy device, the massage therapy device comprising:
a housing provided with one or more holes;
one or more spherical structures provided partially within the one or more respective holes of the housing, wherein the one or more spherical structures are configured to rotate with respect to the housing, while remaining attached within the housing; and
a plurality of stimulation elements disposed along outer surfaces of the one or more spherical structures, wherein the plurality of stimulation elements are directed in an outward direction from the outer surfaces of the one or more spherical structures.

2. The massage therapy device as claimed in claim 1, wherein the housing is configured to receive power through wired or wireless means from a power source.

3. The massage therapy device as claimed in claim 2, wherein the housing includes a wireless charging coil and the one or more spherical structures include one or more respective induction coils, the one or more induction coils being configured to receive power at any angle or orientation through coupling with the electromagnetic field generated by the wireless charging coil due to flow of electric current in the wireless charging coil.

4. The massage therapy device as claimed in claim 3, wherein the one or more spherical structures include one or more respective batteries configured to be charged through the power received by one or more respective induction coils.

5. The massage therapy device as claimed in claim 3, wherein the power received by the one or more induction coils is used to activate the plurality of stimulation elements of the one or more spherical structures.

6. The massage therapy device as claimed in claim 3, wherein the one or more spherical structures include one or more respective balancing devices configured to align the one or more respective induction coils with the wireless charging coil of the housing when the massage therapy device is in a stationary position.

7. The massage therapy device as claimed in claim 1, wherein the plurality of stimulation elements include one or more of Light Emitting Diodes (LEDs), heating elements, cooling elements, vibration elements, electrodes and combinations thereof.

8. The massage therapy device as claimed in claim 7, wherein the LEDs are configured to emit radiation in visible light and infrared frequencies of the electromagnetic spectrum.

9. The massage therapy device as claimed in claim 8, wherein the wavelengths of the light emitted by the LEDs lie in the ranges of 380 nm to 700 nm for the visible light and 845 nm to 855 nm for the infrared light.

10. The massage therapy device as claimed in claim 7, wherein the one or more spherical structures are made up of himalayan salt thereby allowing radiation from the LEDs to pass through.

11. The massage therapy device as claimed in claim 1, further comprising one or more gyroscope sensors provided within the one or more respective spherical structures.

12. The massage therapy device as claimed in claim 11, wherein the gyroscope sensor in each ball is configured to disconnect power supply to the plurality of stimulation elements that are directed towards the ambient, when the massage therapy device is in use.

13. The massage therapy device as claimed in claim 1, wherein the one or more spherical structures are removably attached with the housing and are replaceable with respect to the housing.

14. The massage therapy device as claimed in claim 1, further comprising a control unit configured to control the operation of the plurality of stimulation elements in response to an input received from an external communication device.

15. A method of manufacturing a massage therapy device, the method comprising:
providing a housing with one or more holes;
providing, partially, one or more spherical structures in the one or more respective holes, wherein the one or more spherical structures are configured to rotate with respect to the housing, while remaining attached within the housing;
disposing a plurality of stimulation elements along outer surfaces of the one or more spherical structures, wherein the plurality of stimulation elements are directed in an outward direction from the outer surfaces of the one or more spherical structures.
